(19) United States
(12) Patent Application Publication
(10) Pub. No.: US 2025/0320390 A1
(43) Pub. Date: Oct. 16, 2025

(54) COMPOSITE STRUCTURAL ADHESIVE COMPOSITIONS AND RELATED METHODS

(71) Applicant: INSIGHTEC LTD., Tirat Carmel (IL)

(72) Inventors: Yonatan SHACHAF, Tirat Carmel (IL); Oleg PRUS, Haifa (IL); Vladislav DEMENSKY, Kiryat Ata (IL); Adi GREENBERG, Haifa (IL)

(21) Appl. No.: 18/865,661

(22) PCT Filed: May 13, 2023
(86) PCT No.: PCT/IB2023/054951

§ 371 Date: Nov. 13, 2024

Related U.S. Application Data
(60) Provisional application No. 63/341,556, filed on May 13, 2022.

(51) Int. Cl. C09J 163/00; A61N 7/02; B06B 1/06; C08K 3/34; C08K 3/40; C08K 7/26; C09J 5/00; C09J 11/04

(52) U.S. Cl. CPC C09J 163/00; A61N 7/02; B06B 1/0607; C09J 5/00; C09J 11/04; C08K 2003/343; C08K 3/40; C08K 7/26; C09J 2400/166; C09J 2463/00

(57) ABSTRACT

The various implementations described herein include adhesive compositions for high-voltage focused ultrasound applications. In one aspect, an ultrasound transducer comprises a plurality of tiles. Each tile includes a first electrically conductive layer, a second electrically conductive layer, a plurality of transducer elements disposed between the first electrically conductive layer and the second electrically conductive layer, and a composite structural adhesive (a) disposed between the first electrically conductive layer and the second electrically conductive layer and (b) disposed between respective adjacent transducer elements of the plurality of transducer elements. The composite structural adhesive includes an epoxy resin, fumed silica, and a powder selected from the group consisting of: (i) micronized mica, (ii) bioactive glass powder, and (iii) micronized mica and bioactive glass powder.

### RELATED APPLICATIONS

This application claims priority to U.S. Provisional Patent Application No. 63/341,556, filed on May 13, 2022, which is incorporated by reference herein in its entirety.

### TECHNICAL FIELD

The disclosure relates generally to adhesive compositions, and more particularly to composite structural adhesives for high-voltage focused ultrasound applications.

### BACKGROUND

Focused ultrasound (i.e., acoustic waves having a frequency greater than about 20 kilohertz) can be used to image or therapeutically treat internal body tissues within a patient. For example, ultrasound waves may be used in applications involving ablation of tumors, thereby eliminating the need for invasive surgery, targeted drug delivery, control of the blood-brain barrier, lysing of clots, neuromodulation and other clinical procedures. During tumor ablation, an ultrasound transducer is placed externally to the patient, but in close proximity to the tissue to be ablated (i.e., the target). The ultrasound transducer converts an electronic drive signal into mechanical vibrations, resulting in the emission of acoustic waves. The transducer may be geometrically shaped and positioned along with other such transducers so that the ultrasound energy they emit collectively forms a focused beam at a “focal zone” corresponding to (or within) the target tissue region. Alternatively or additionally, a single transducer may be formed of a plurality of individually driven transducer elements whose phases can each be controlled independently. Such a “phased-array” transducer facilitates steering the focal zone to different locations by adjusting the relative phases among the transducers. As used herein, the term “element” means either an individual transducer in an array or an independently drivable portion of a single transducer. In some instances, magnetic resonance imaging (MRI) may be used to visualize the patient and target, and thereby to guide the ultrasound beam.

Conventional ultrasound transducers are multilayer structures laminated in a stacked configuration between two electrode layers for providing high power-delivery efficiency. The number and order of the layers and their thicknesses may be selected based on the desired frequency-response profile. Typically, at least one of the layers is formed of a piezoelectric (e.g., piezoceramic) material that can be driven by electric signals to produce ultrasound energy. Other intervening layers contribute to mechanical stability and efficient delivery of the energy. Because each layer may have different acoustic parameters, a combination of layers can generate a desired working frequency response with suitable bandwidth. But maintaining the overall mechanical integrity of the transducer structure, particularly preventing delamination of the layers over time, can be challenging.

An exemplary transducer structure includes three intervening layers: resin-impregnated graphite, lead zirconate titanate (PZT), which is piezoelectric, and copper-impregnated graphite. Operating voltages depend on the necessary acoustic power delivery. For imaging applications, an ultrasound transducer may be driven at tens of volts; for therapeutic applications requiring the delivery of high acoustic power levels to disrupt or ablate tissue, the transducer may be driven at hundreds of volts. Some therapeutic applications, such as histotripsy, may drive transducers at thousands of volts in order to achieve very high instantaneous power levels and cause cavitation at an internal anatomic target.

In general, an adhesive for bonding transducer elements should exhibit good mechanical properties, a high glass transition temperature (Tg), and amenability to coating using, for example, an electroless copper plating process. For high-voltage focused ultrasound applications, however, the adhesive must also be able to tolerate the resulting stresses and at the same time provide enough electrical isolation to prevent arcing across bonded elements. Many adhesives that satisfy the basic performance criteria fail the latter requirement. Accordingly, there is a need for adhesive compositions that may be applied using conventional techniques yet provide high strength, durability, and strong electrical isolation.

### SUMMARY

The present disclosure is directed to composite structural adhesives for high-voltage focused ultrasound applications. The composite structural adhesives comprise epoxy and a filler that includes an amorphous powder and fumed silica. They exhibit good mechanical properties following cure, a high glass transition temperature, and amenability to coating using common techniques.

In various implementations of the present disclosure, the composite structural adhesives described herein: (i) have sufficient heat deflection temperature (HDT) of at least 120° C.; (ii) structural stability during manufacturing and operation (e.g., at >3000V) of the transducer; (iii) adhesion to copper (e.g., via a plating process, such as electroless plating process or an electroplating process); (iv) high electrical resistance; and/or (v) zero or minimal interference with electrical and acoustical impedance of the piezoelectric material

In some implementations, an ultrasound transducer comprises a plurality of tiles, each tile comprising a first electrically conductive layer, a second electrically conductive layer, a plurality of transducer elements disposed (e.g., sandwiched) between the first electrically conductive layer and the second electrically conductive layer, and a composite structural adhesive. In some implementations, the composite structural adhesive is: (a) disposed (e.g., sandwiched) between the first electrically conductive layer and the second electrically conductive layer, and (b) disposed between respective adjacent transducer elements of the plurality of transducer elements. In some implementations, the composite structural adhesive includes an epoxy resin, fumed silica, and a powder selected from the group consisting of: (i) micronized mica, (ii) bioactive glass powder, and (iii) micronized mica and bioactive glass powder.

In some implementations, the composite structural adhesive is in direct contact with the first electrically conductive layer and the second electrically conductive layer. In some implementations, the plurality of transducer elements forms a curved array of transducer elements. In some implementations, the respective transducer elements in the plurality of transducer elements are separated by a first spacing. In some implementations, the first spacing does not exceed 1 mm. In some implementations, the first spacing does not exceed 500 μm. In some implementations, the first spacing does not exceed 300 μm. In some implementations, the powder comprises particles having a maximum diameter not exceeding the first spacing. In some implementations, the powder comprises particles having an average diameter not exceeding 300 μm. In some implementations, each of the first electrically conductive layer and the second electrically conductive layer is formed by electroplating. In some implementations, each of the first electrically conductive layer and the second electrically conductive layer is formed by electroless electroplating. In some implementations, each of the first electrically conductive layer and the second electrically conductive layer comprises a copper layer.

In various implementations of the present disclosure, a composite structural adhesive comprises an epoxy resin, fumed silica, and a powder selected from the group consisting of: (i) micronized mica, (ii) bioactive glass powder, and (iii) micronized mica and bioactive glass powder. In some implementations, the composite structural adhesive is used for focused ultrasound applications.

In some implementations, the powder consists of micronized mica. In some implementations, the powder consists of bioactive glass powder. In some implementations, the powder consists of micronized mica and bioactive glass powder. In some implementations, the powder comprises particles having an average diameter not exceeding 300 μm. In some implementations, the epoxy resin is bisphenol A diglycidyl ether epoxy. In some implementations, the epoxy resin has a glass transition temperature (Tg) of 90° C. after thermal curing. In some implementations, the epoxy resin is a combination of hardener and resin that yields a threshold Tg of 90° C. In some implementations, following cure, the adhesive exhibits heat deflection (e.g., heat deflection temperature) to at least 120° C.

In various implementations of the present disclosure, a method of manufacturing an ultrasound transducer tile comprises casting an epoxy mixture on a piezoelectric ceramic tile to form a casted component. The epoxy mixture comprises an epoxy resin, fumed silica, and a powder. The powder consists essentially of (i) micronized mica, (ii) bioactive glass powder, or (iii) micronized mica and bioactive glass powder. The method comprises curing the casted component to form a cured component. The method comprises plating a copper layer over the cured component.

In some implementations, the method further comprises, while the casted component is curing, pressing the casted component into a curved shape. In some implementations, the method further comprises, after plating the copper layer over the cured component, operating the ultrasound transducer tile at an operating voltage of 3000V. In some implementations, the method further comprises, after plating the copper layer over the cured component, assembling the ultrasound transducer tile into a single multi-tile transducer array comprising a plurality of ultrasound transducer tiles.

Thus, composite structural adhesives for high-voltage focused ultrasound applications are provided.

Note that the various implementations described above can be combined with any other implementations described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the disclosure. In the following description, various implementations of the present disclosure are described with reference to the following drawings, in which:

FIG. 1A schematically depicts an exemplary ultrasound system in accordance with various implementations of the current disclosure.

FIG. 1B schematically depicts an exemplary MRI system in accordance with various implementations of the current disclosure.

FIG. 2 depicts an implementation of an acoustic reflector substantially close to a target region in accordance with some implementations.

FIGS. 3A and 3B illustrate a transducer tile, in accordance with some implementations.

FIG. 3C illustrates exemplary processes of manufacturing a curved array of transducer elements in accordance with some implementations.

FIG. 4 details the results of various formulation experiments in accordance with some implementations of the present disclosure.

FIG. 5 shows the chemical and physical properties of micronized mica that were used in the experiments described with reference to FIG. 4.

FIG. 6 illustrates experimental details for optimizing the mica and CABOSIL filler formulation to achieve satisfactory copper adhesion, in accordance with some implementations of the present disclosure.

FIG. 7 illustrates the conversion to weight (w/w) percentage in the overall epoxy formulation based on the average of four separate measurements.

FIG. 8 illustrates a flowchart for a method of manufacturing an ultrasound transducer tile in accordance with some implementations.

### DETAILED DESCRIPTION

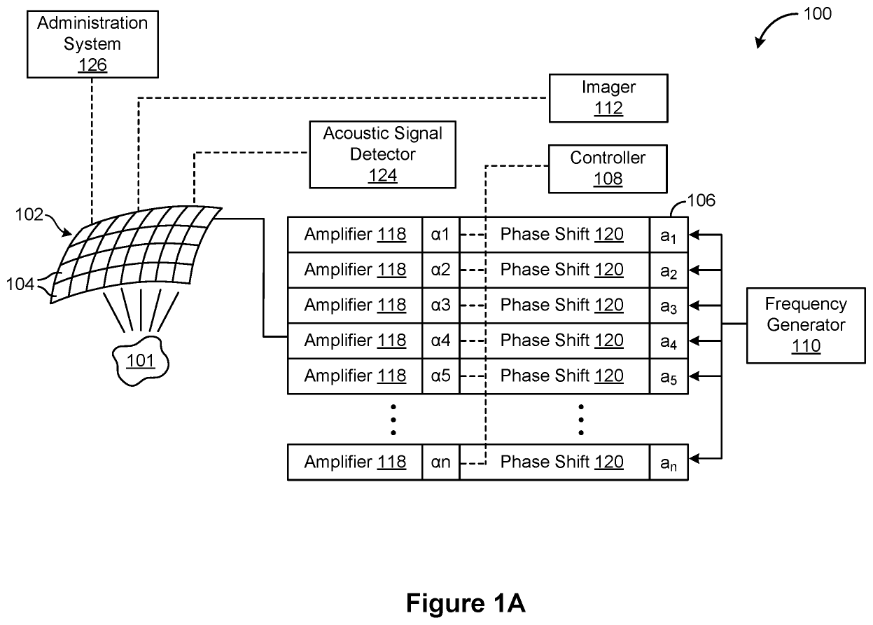

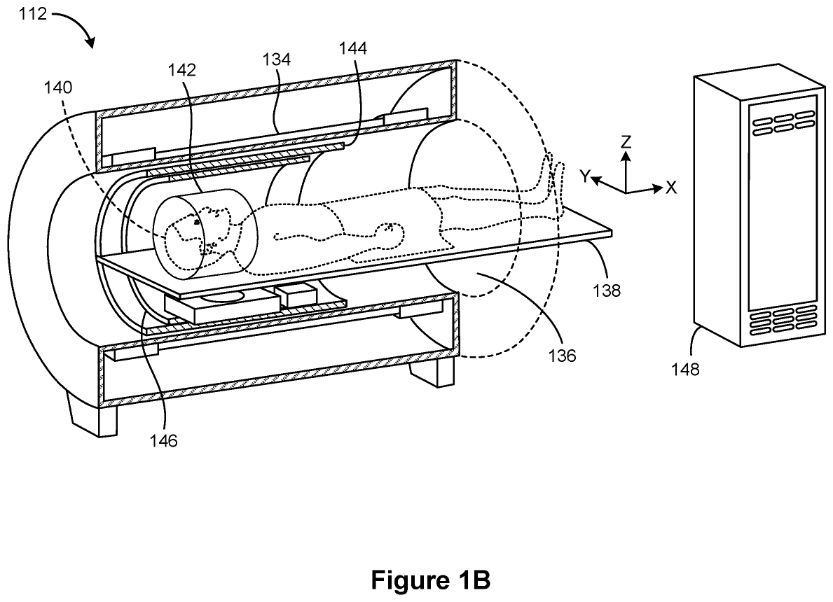

FIG. 1A illustrates an exemplary ultrasound system 100 for generating and delivering a focused acoustic energy beam to a target region 101 within a patient's body. The illustrated system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) or other contoured shape suitable for placement on the surface of the patient's body, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50 (2, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each including or consisting of an amplifier 118 and a phase delay circuit 120; each drive circuit drives one of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some implementations, the frequency generator 110 is integrated with the beamformer 106. The radio frequency generator 110 and the beamformer 106 are configured to drive the individual transducer elements 104 of the transducer array 102 at the same frequency, but at different phases and/or different amplitudes.

The amplification or attenuation factors α₁-αn and the phase shifts a₁-an imposed by the beamformer 106 serve to transmit and focus ultrasonic energy through the intervening tissue located between the transducer elements 104 and the target region onto the target region 101, and account for wave distortions induced in the intervening tissue. The amplification factors and phase shifts are computed using the controller 108, which may provide the computational functions through software, hardware, firmware, hardwiring, or any combination thereof. In various implementations, the controller 108 utilizes a general-purpose or special-purpose digital data processor programmed with software in a conventional manner, and without undue experimentation, to determine the frequency, phase shifts and/or amplification factors necessary to obtain a desired focus or any other desired spatial field patterns at the target region 101. In certain implementations, the computation is based on detailed information about the characteristics (e.g., the type, size, location, property, structure, thickness, density, structure, etc.) of the intervening tissue located between the transducer element 104 and the target and their effects on propagation of acoustic energy. Such information may be obtained from an imager 112. The imager 112 may be, for example, a magnetic resonance imaging (MRI) device, a computer tomography (CT) device, a positron emission tomography (PET) device, a single-photon emission computed tomography (SPECT) device, or an ultrasonography device. Image acquisition may be three-dimensional (3D) or, alternatively, the imager 112 may provide a set of two-dimensional (2D) images suitable for reconstructing a three-dimensional image of the target region 101 and/or other regions (e.g., the region surrounding the target 101 or another target region). Image-manipulation functionality may be implemented in the imager 112, in the controller 108, or in a separate device. In some implementations, the ultrasound system 100 and/or imager 112 may be utilized to detect signals from an acoustic reflector (e.g., microbubbles) located substantially close to the target region 101. Additionally or alternatively, the system 100 may include an acoustic-signal detection device (such as a hydrophone or suitable alternative) 124 that detects transmitted or reflected ultrasound from the acoustic reflector, and which may provide the signals it receives to the controller 108 for further processing. In addition, the ultrasound system 100 may include an administration system 126 for parenterally introducing the acoustic reflector into the patient's body. The imager 112, the acoustic-signal detection device 124, and/or the administration system 126 may be operated using the same controller 108 that facilitates the transducer operation; alternatively, they may be separately controlled by one or more separate controllers intercommunicating with one another.

FIG. 1B illustrates an exemplary imager-namely, an MRI apparatus 112. The apparatus 112 may include a cylindrical electromagnet 134, which generates the requisite static magnetic field within a bore 136 of the electromagnet 134. During medical procedures, a patient is placed inside the bore 136 on a movable support table 138. A region of interest 140 within the patient (e.g., the patient's head) may be positioned within an imaging region 142 wherein the electromagnet 134 generates a substantially homogeneous field. A set of cylindrical magnetic field gradient coils 144 may also be provided within the bore 136 and surrounding the patient. The gradient coils 144 generate magnetic field gradients of predetermined magnitudes, at predetermined times, and in three mutually orthogonal directions. With the field gradients, different spatial locations can be associated with different precession frequencies, thereby giving a magnetic-resonance (MR) image its spatial resolution. An RF transmitter coil 146 surrounding the imaging region 142 emits RF pulses into the imaging region 142 to cause the patient's tissues to emit MR response signals. Raw MR response signals are sensed by the RF coil 146 and passed to an MR controller 148 that then computes an MR image, which may be displayed to the user. Alternatively, separate MR transmitter and receiver coils may be used. Images acquired using the MRI apparatus 112 may provide radiologists and physicians with a visual contrast between different tissues and detailed internal views of a patient's anatomy that cannot be visualized with conventional x-ray technology.

The MRI controller 148 may control the pulse sequence, i.e., the relative timing and strengths of the magnetic field gradients and the RF excitation pulses and response detection periods. The MR response signals are amplified, conditioned, and digitized into raw data using a conventional image-processing system, and further transformed into arrays of image data by methods known to those of ordinary skill in the art. Based on the image data, the target region (e.g., a tumor, a clot, a specific brain tissue, or a target BBB) can be identified.

To perform targeted drug delivery or a tissue or tumor ablation, it is necessary to determine the location of the target region 101 with high precision. Accordingly, in various implementations, the imager 112 is first activated to acquire images of the target region 101 and/or non-target region (e.g., the healthy tissue surrounding the target region, the intervening tissue located between the transducer array 102 and the target region 101 and/or any regions located near the target) and, based thereon, determine anatomical characteristics (e.g., the tissue type, location, size, thickness, density, structure, shape, vascularization) associated therewith. For example, a tissue volume may be represented as a 3D set of voxels based on a 3D image or a series of 2D image slices and may include the target region 101 and/or non-target region.

To create a high-quality focus at the target region 101, it may be necessary to calibrate the transducer elements 104 and take into account transducer geometric imperfections resulting from, for example, movement, shifts and/or deformation of the transducer elements 104 from their expected locations. In addition, because the ultrasound waves may be scattered, absorbed, reflected and/or refracted when traveling through inhomogeneous intervening tissues located between the transducer elements 104 and the target region 101, accounting for these wave distortions may also be necessary in order to improve the focusing properties at the target region 101.

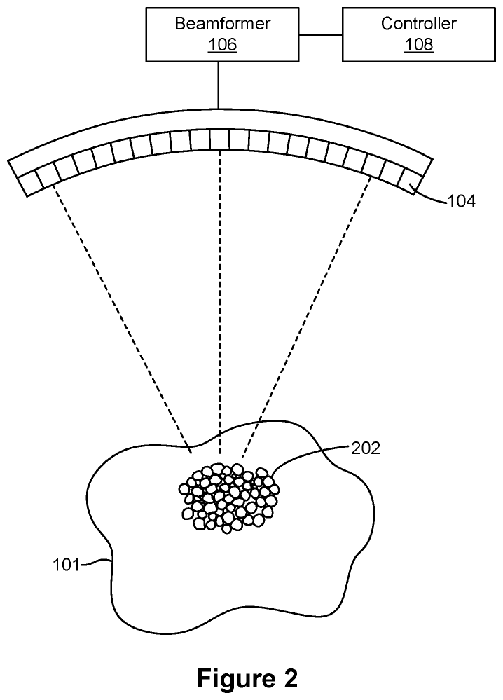

Referring to FIG. 2, in various implementations, calibration of the transducer geometry as well as correction of the beam aberrations caused by the inhomogeneous tissues are facilitated by employing an acoustic reflector 202 substantially close to the target region 101. Ultrasound waves transmitted from all (or at least some) transducer elements 104 are reflected by the reflector 202. The acoustic reflector 202 may consist essentially of microbubbles generated by the ultrasound waves and/or introduced parenterally by an administration system. In some implementations, the administration device 126 introduces a seed microbubble into the target region 101; the transducer 102 is then activated to transmit ultrasound waves to the seed microbubble for generating a cloud of microbubbles. Approaches to generating the microbubbles and/or introducing the microbubbles to the target region 101 are provided, for example, in PCT Publication No. WO 2018/020315, PCT Application Nos. PCT/US2018/064058 (filed on Dec. 5, 2018), PCT/IB2018/001103 (filed on Aug. 14, 2018), PCT/US2018/064892 (filed on Dec. 11, 2018), PCT/IB2018/000841 (filed on Jun. 29, 2018), and PCT/US2018/064066 (filed on Dec. 5, 2018), U.S. Patent Publication No. 2019/0083065, and U.S. patent application Ser. No. 15/837,392 (filed on Dec. 11, 2017), the contents of which are incorporated herein by reference.

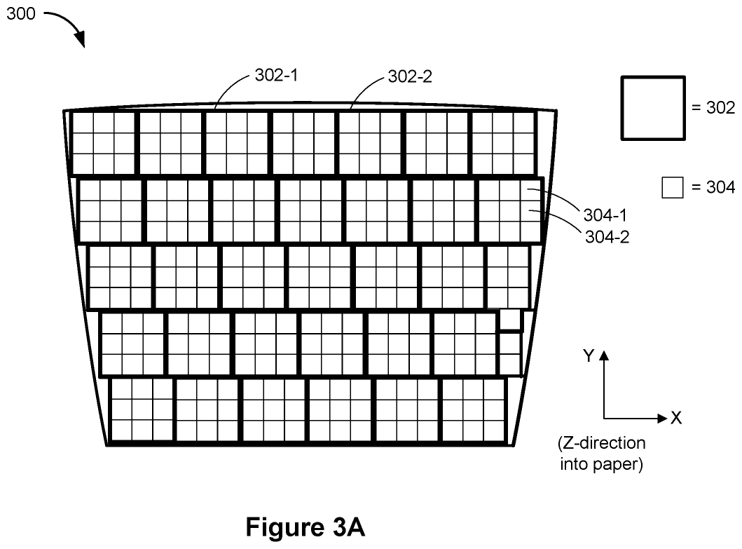

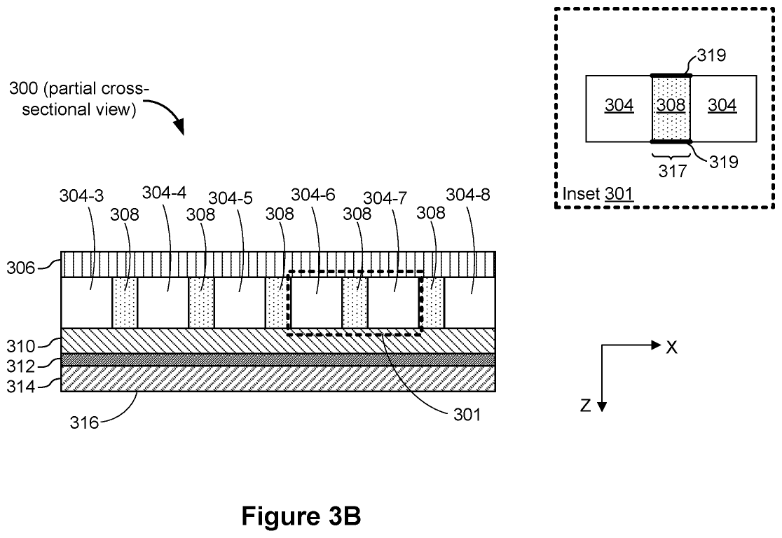

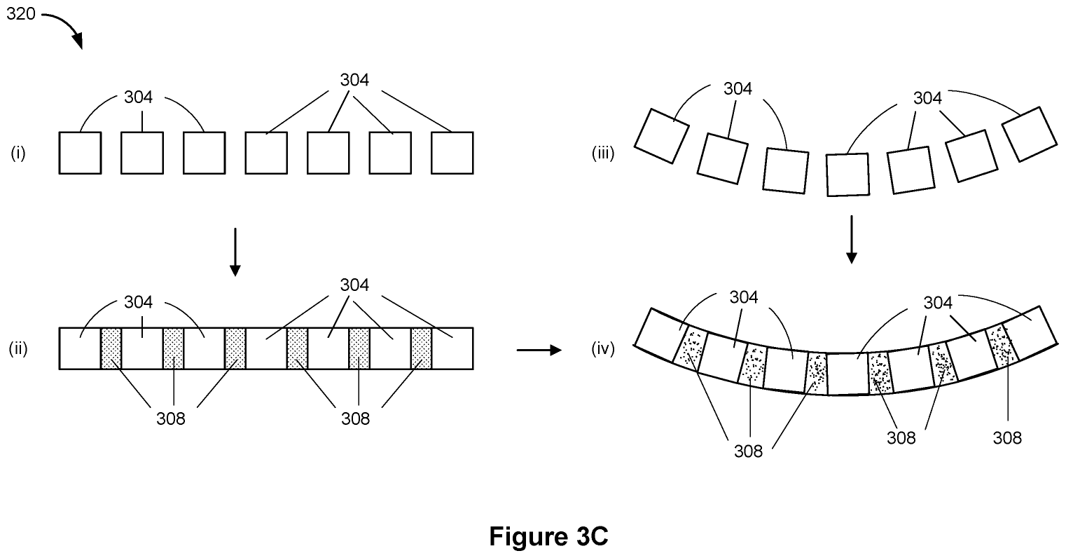

FIG. 3A illustrates an exemplary transducer tile 300 in accordance with some implementations. In some implementations, an ultrasound transducer comprises one or more transducer tiles 300, each tile including a respective number of transducer elements 304 (e.g., transducer element 304-1 and transducer element 304-2). In some implementations, every transducer element 304 in the tile is independently controllable. In some implementations, not every transducer element 304 is independently controllable; instead, the transducer elements 304 in a given group 302 are wired and controllable at the group level (e.g., group 302-1 and group 302-1), and each of the groups 302 is independently wired. In some implementations, a group 302 comprises anywhere between 2 and 10, 2 and 15, 2 and 20, 2 and 25, 2 and 30, or 2 and 50 transducer elements. A respective group 302 includes a closed-form shape that is circular, elliptical, or an N-gon, where N is a value between 1 and 20.

FIG. 3B illustrates a cross-sectional view of a transducer tile 300 or a portion thereof, in accordance with some implementations. The transducer elements 304 (e.g., transducer elements 304-3, 304-4, 304-5, 304-6, 304-7, and 304-8) are disposed (e.g., sandwiched) between a first layer 306 and a second layer 310. Respective (e.g., adjacent) transducer elements are separated by a spacing 317, as shown in inset 301 of FIG. 3B. Depending on the implementation, the spacing 317 does not exceed 2 mm, 1 mm, 500 μm, 300 μm, or 250 μm.

In some implementations, each of the transducer elements 304 is in direct contact with the first layer 306. In some implementations, each of the transducer elements 304 is in direct contact with the second layer 310.

FIG. 3B also shows a composite structural adhesive 308 disposed (e.g., sandwiched) between adjacent transducer elements 304. In some implementations, the composite structural adhesive 308 is in direct contact with the first layer 306. In some implementations, the composite structural adhesive 308 is in direct contact with the second layer 310. In some implementations, the composite structural adhesive 308 is electrically insulative.

In some implementations, the first layer 306 is an electrically conductive layer. In some implementations, the first layer 306 comprises copper. In some implementations, the first layer 306 is formed by electroplating. In some implementations, the first layer 306 is formed by electroless electroplating.

In some implementations, the second layer 310 is an electrically conductive layer. In some implementations, the second layer 310 comprises copper. In some implementations, the second layer 310 is formed by electroplating. In some implementations, the second layer 310 is formed by electroless electroplating.

FIG. 3B also shows that in some implementations, the transducer tile 300 includes a matching layer 314 below the second layer 310. In some implementations, the matching layer 314 includes graphite and is glued to the second layer 310 using an electrically conductive glue 312 that, in some implementations, includes graphite. In some implementations, the other side of the matching layer (surface 316) is connected to a grounding layer with an electrically conductive adhesive.

FIG. 3C illustrates exemplary processes 320 of manufacturing a curved array of transducer elements 304 in accordance with some implementations.

In some implementations, the process begins at step (i) and continues to steps (ii) and (iv). The process starts by arranging a plurality of transducer elements 304 on a planar surface, as shown in step (i). The composite structural adhesive 308 is cast on the transducer elements 304 to form a casted array of transducer elements, as shown in step (ii). The process includes forming the casted array into a curved array as shown in step (iv), (e.g., by pressing the casted array into a curved shape) while the casted array is curing.

In some implementations, the process begins at step (iii) and continues to step (iv). In these implementations, the process starts by arranging a plurality of transducer elements 304 on a curved surface, as depicted in step (iii). The composite structural adhesive 208 is cast on the transducer elements 304 while they are on the curved surface to form a casted curved array, as depicted in step (iv), and the curved array proceeds to cure.

In some implementations, the composite structural adhesive 308 comprises an electrically insulative adhesive. In some implementations, the composite structural adhesive 308 includes epoxy and a filler. In some implementations, the filler comprises or consists essentially of an amorphous powder and fumed silica.

In some implementations, the amorphous powder may be bioactive glass or micronized mica. The term “micronized mica” herein refers generally to mica (a phyllosilicate mineral) in powder form. Depending on the implementation, the amorphous powder may be one or more of: (i) mica (e.g., 325 Mesh mica) (e.g., with particle sizes of ˜44 μm); (ii) bioactive glass (e.g., VITRYXX bioactive glass product MD01); or (iii) a combination of mica and bioactive glass (e.g., VITRYXX product G018-270).

In some implementations, the fumed silica is hydrophilic (e.g., CABOSIL hydrophilic fumed silica). The fumed silica improves the wettability (e.g., the ability of a liquid to spread over a surface) of surfaces 319 (FIG. 3B inset 301), which facilitates coating of the first layer 306 and/or the second layer 310 over the surface(s) of the composite structural adhesive 308.

In some implementations, while there may be no fixed lower limit to acceptable powder sizes, a practical upper limit may be determined by the spacing 317 (e.g., gap) between adjacent transducer elements 304 that the composite structural adhesive 308 fills. For example, depending on the implementations, the powder may comprise particles having a maximum diameter that does not exceed 2 mm, 1 mm, 500 μm, 300 μm, or 250 μm. Further, depending on the implementation, the powder may comprise particles having an average diameter not exceeding 2 mm, 1 mm, 500 μm, 300 μm, or 250 μm.

The choice of epoxy for the composite structural adhesive 308 may be determined for a particular application based on a threshold glass transition temperature Tg (optionally, safety margins may be applied by slightly increasing the Tg). For example, for sonication procedures during which target tissue (e.g., brain tissue or uterine fibroids) is thermally ablated by being subjected to continuous ultrasound energy, the focused ultrasound transducer may reach 90-110° C. when voltage is applied. For such implementations, the epoxy resin may be a combination of hardener and resin that yields a threshold Tg of at least 90° C., 95° C., 100° C., or 105° C. In principle, any combination of hardener and resin that yields the threshold Tg (e.g., at least 90° C., 95° C., 100° C., or 105° C.) can potentially be used. Bisphenol A diglycidyl ether epoxy (also known as diglycidyl ether of bisphenol A (DGEBA)) resin, used with different hardeners during development, may be adequate for applications requiring heat deflection to approximately 120° C. Hardeners (e.g., adduct hardeners or other types of hardeners), which contain a small amount of the epoxy resin, may be employed. An adduct hardener can be any suitable amine hardener-monoamine, diamine, tri-amine, or polyamine, etc. Suitable amine hardeners include ethyleneamines (e.g., diethylenetriamine, triethylenetetramine, tetraethylenepentamine, and N-aminoethylpiperazine), cycloaliphatics (e.g., bis-(p-aminocyclohexyl)methane, diaminocyclohexane, and bis-(dimethyldiaminocyclohexyl)methane), and aromatics (e.g., methylene dianiline, m-phenylene diamine, and diaminophenyl sulfone).

In practice, the mica and fumed silica may be mixed with the resin, degassed and then mixed with the hardener. Additional degassing may be performed when the epoxy is cast. The epoxy resin combined with the mica and fumed silica filler may be stored for long periods of time, and may be mixed before use to yield a homogeneous fluid for casting.

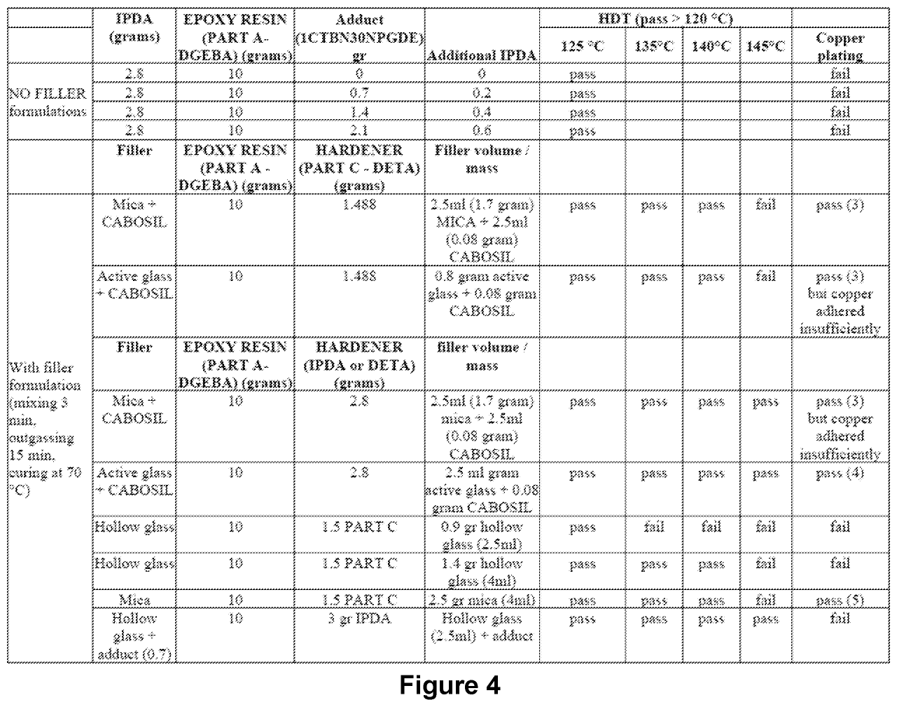

FIG. 4 details the results of various formulation experiments in accordance with some implementations of the present disclosure. As shown in FIG. 4, experiments were conducted with “no filler formulations” (i.e., epoxy only) and “filler formulations” that include micronized mica (“mica” in FIG. 4), bioactive glass (“active glass” in FIG. 4) or both mica and active glass. Control experiments were performed using hollow glass and adduct (see data in the last row of FIG. 4). The adduct hardener was isophorone diamine (IPDA) or “PART C,” which refers to diethylenetriamine (DETA). “Active glass” refers to VITRYXX bioactive glass.

For the formulation experiments, each formulation was tested for heat deflection temperature (HDT) and copper plating adhesion. The copper plating adhesion tests were performed by adhering Kapton tape onto the first and second layers 306 and 310 (e.g., copper coating layers), followed by removal of the Kapton tape from the first and second layers and inspecting the tape for signs of copper removal. The last column in FIG. 4 shows the results of the copper plating adhesion tests. The numerals following “pass” entries indicate the relative quality of the copper coating, with higher values corresponding to better quality.

As noted in the last data row in FIG. 4, hollow glass (which has the same chemical formula as active glass) did not produce formulations with satisfactory results. A possible reason is that Hollow glass is composed of spherical particles (e.g., the H20 hollow glass microsphere product supplied by Sinosteel Maanshan New Material Technology, Maanshan, China) and do not present sufficient surface roughness to afford adequate copper adhesion to the epoxy. Bioactive glass, by contrast—like mica—may have a significantly higher degree of surface roughness that promotes good copper adhesion through interdigitation.

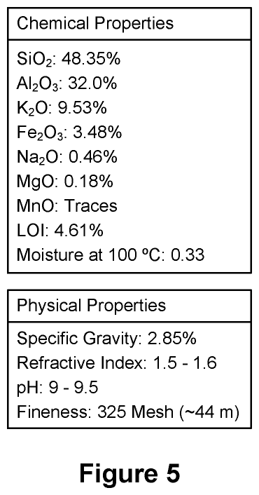

FIG. 5 shows the chemical and physical properties of micronized mica that were used in the experiments described with reference to FIG. 4. As shown in FIG. 5, the average specifications of micronized mica corresponding to physical properties such as specific gravity of 2.85%, refractive index of 1.5-1.6, pH of 9-9.5, and fineness of 325 mesh.

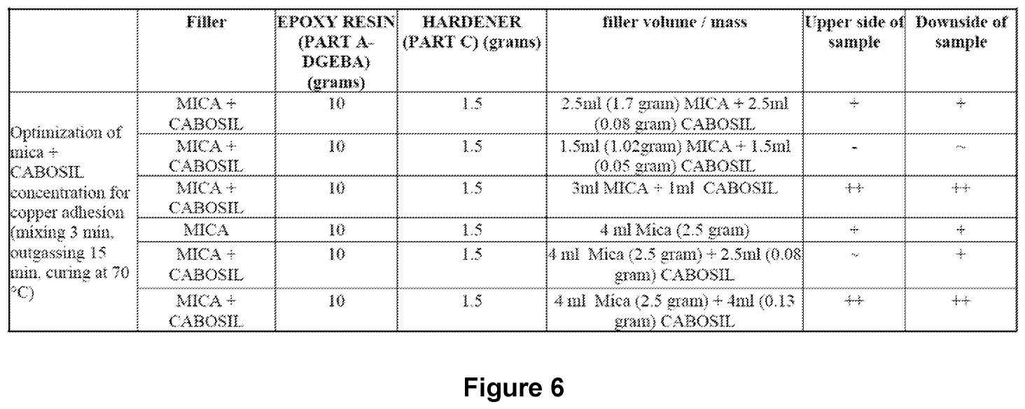

FIG. 6 illustrates experimental details for optimizing the mica and CABOSIL (e.g., fumed silica) filler formulation to achieve satisfactory copper adhesion, in accordance with some implementations of the present disclosure.

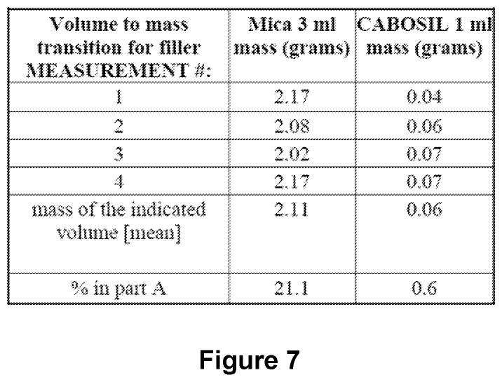

As can be seen from FIG. 4, various formulations including micronized mica, bioactive glass or both types of powder exhibit high HDT and satisfactory copper adhesion. A filler composition including 3 mL of mica and 1 mL of CABOSIL produced optimal results as indicated in FIG. 6. FIG. 7 illustrates the conversion to weight (w/w) percentage in the overall epoxy formulation based on the average of four separate measurements.

More generally, 10.55-28% w/w micronized mica in the epoxy formulation were found to enable sufficient adhesion to electroplated copper. The CABOSIL helps control the viscosity of the epoxy, preventing precipitation of the powder during curing. It has minimal effect on copper plating and may therefore be added generously (up to 12% w/w in the epoxy formulation). Excessive viscosity, however, can interfere with a production process if too high. For example, levels of CABOSIL above 0.6% w/w in the uncured epoxy may produce viscosities too high for common production sequences.

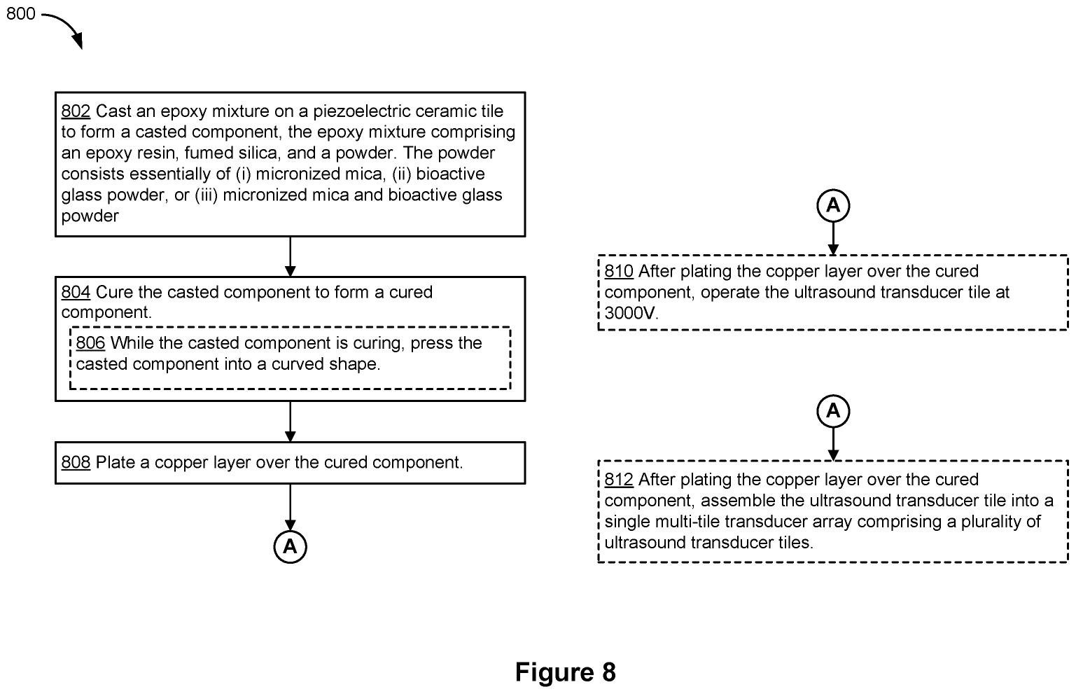

FIG. 8 illustrates a flowchart for a method 800 of manufacturing an ultrasound transducer tile, in accordance with some implementations. The ultrasound transducer tile may be manufactured by machining.

The method includes casting (802) an epoxy mixture on a piezoelectric ceramic tile to form a casted component. The epoxy mixture comprises an epoxy resin, fumed silica, and a powder. The powder consists essentially of (i) micronized mica, (ii) bioactive glass powder, or (iii) micronized mica and bioactive glass powder. In some implementations, the mixture of epoxy resin and filler (fumed silica and powder) is combined with the hardener and cast on the machined tile in a vacuum chamber (desiccator). After 15 minutes of degassing inside the chamber, the tile is removed from the desiccator and pressed to form the required curvature. During pressing, the tile and epoxy are subjected to thermal curing (4 hours at room temperature following 6 hours at 75° C.).

The method 800 includes curing (804) the casted component to form a cured component.

In some implementations, the method includes, while the casted component is curing, pressing (806) the casted component into a curved shape.

In some implementations, the method includes plating (808) a copper layer over the cured component. In some implementations, the cured surface of the piezoelectric ceramic tile (cured component) is then prepared for plating by stone polishing following sand blasting and acid etching. One or more layers of copper are applied by electroless plating to a typical thickness of 3 μm. The plated tile may then be subjected to post-curing at 120° C. for 40 min after it is glued to the graphite matching layer (e.g., matching layer 314). Multiple tiles prepared in accordance with this sequence may then be assembled into a single multi-tile transducer array and wired.

In some implementations, the method includes, after plating the copper layer over the cured component, operating (810) the ultrasound transducer tile at 3000 V. In some implementations, the ultrasound tile is configured to operate at voltages of up to 3000 V or higher.

In some implementations, the method includes, after plating the copper layer over the cured component, assembling (812) the ultrasound transducer tile into a single multi-tile transducer array comprising a plurality of ultrasound transducer tiles.

The terms and expressions employed herein are used as terms and expressions of description and not of limitation, and there is no intention, in the use of such terms and expressions, of excluding any equivalents of the features shown and described or portions thereof. In addition, having described certain implementations of the disclosure, it will be apparent to those of ordinary skill in the art that other implementations incorporating the concepts disclosed herein may be used without departing from the spirit and scope of the disclosure. Accordingly, the described implementations are to be considered in all respects as only illustrative and not restrictive

As used herein, the term “substantially” means ±10%, and in some implementations, ±5%. Reference throughout this specification to “one example,” “an example,” “one implementation,” or “an implementation” means that a particular feature, structure, or characteristic described in connection with the example is included in at least one example of the present technology. Thus, the occurrences of the phrases “in one example,” “in an example,” “one implementation,” or “an implementation” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

In addition, the term “controller” used herein broadly includes all necessary hardware components and/or software modules utilized to perform any functionality as described above; the controller may include multiple hardware components and/or software modules and the functionality can be spread among different components and/or modules.

Certain implementations of the present disclosure are described above. It is, however, expressly noted that the present disclosure is not limited to those implementations; rather, additions and modifications to what is expressly described herein are also included within the scope of the disclosure.

1.-30. (canceled)

31. An ultrasound transducer comprising a plurality of tiles, each tile including:
a first electrically conductive layer;
a second electrically conductive layer;
a plurality of transducer elements disposed between the first electrically conductive layer and the second electrically conductive layer; and
a composite structural adhesive (a) disposed between the first electrically conductive layer and the second electrically conductive layer and (b) disposed between respective adjacent transducer elements of the plurality of transducer elements, the composite structural adhesive including:
an epoxy resin;
fumed silica; and
a powder selected from the group consisting of: (i) micronized mica, (ii) bioactive glass powder, and (iii) micronized mica and bioactive glass powder.

32. The ultrasound transducer of claim 31, wherein the composite structural adhesive is in direct contact with the first electrically conductive layer and the second electrically conductive layer.

33. The ultrasound transducer of claim 31, wherein the plurality of transducer elements forms a curved array of transducer elements.

34. The ultrasound transducer of claim 31, wherein the respective transducer elements in the plurality of transducer elements are separated by a first spacing.

35. The ultrasound transducer of claim 34, wherein the first spacing does not exceed 500 μm.

36. The ultrasound transducer of claim 34, wherein the powder comprises particles having a maximum diameter not exceeding the first spacing.

37. The ultrasound transducer of claim 31, wherein the powder comprises particles having an average diameter not exceeding 300 μm.

38. The ultrasound transducer of claim 31, wherein each of the first electrically conductive layer and the second electrically conductive layer is formed by electroplating.

39. The ultrasound transducer of claim 31, wherein each of the first electrically conductive layer and the second electrically conductive layer is formed by electroless electroplating.

40. The ultrasound transducer of claim 31, wherein each of the first electrically conductive layer and the second electrically conductive layer comprises a copper layer.

41. A composite structural adhesive, comprising:
an epoxy resin;
fumed silica; and
a powder selected from the group consisting of: (i) micronized mica, (ii) bioactive glass powder, and (iii) micronized mica and bioactive glass powder.

42. The composite structural adhesive of claim 41, wherein the powder consists of one of:
micronized mica; or
bioactive glass powder; or
micronized mica and bioactive glass powder.

43. The composite structural adhesive of claim 41, wherein the epoxy resin is bisphenol A diglycidyl ether epoxy.

44. The composite structural adhesive of claim 41, wherein the epoxy resin is a combination of hardener and resin that yields a threshold glass transition temperature (Tg) of 90° C.

45. The composite structural adhesive of claim 41, wherein, following cure, the adhesive exhibits a heat deflection temperature of at least 120° C.

46. A method of manufacturing an ultrasound transducer tile, the method comprising:
casting an epoxy mixture on a piezoelectric ceramic tile to form a casted component, the epoxy mixture comprising an epoxy resin, fumed silica, and a powder, the powder consisting essentially of (i) micronized mica, (ii) bioactive glass powder, or (iii) micronized mica and bioactive glass powder;
curing the casted component to form a cured component; and
plating a copper layer over the cured component.

47. The method of claim 46, further comprising, while the casted component is curing, pressing the casted component into a curved shape.

48. The method of claim 46, further comprising:
after plating the copper layer over the cured component, operating the ultrasound transducer tile at an operating voltage of 3000 V.

49. The method of claim 46, further comprising:
after plating the copper layer over the cured component, assembling the ultrasound transducer tile into a single multi-tile transducer array comprising a plurality of ultrasound transducer tiles.

50. The method of claim 46, wherein the powder consists of micronized mica and bioactive glass powder.
